(19) United States
(12) Patent Application Publication
(10) Pub. No.: US 2020/0101010 A1
(43) Pub. Date: Apr. 2, 2020

(54) SUBLINGUAL FORMULATION WITH WATER-SOLUBLE COCRYSTALS OF ACETYLSALICYLIC ACID WITH CITRIC ACID, SODIUM BICARBONATE, AND L-THEANINE FOR THE TREATMENT OF ACUTE MYOCARDIAL INFARCTION

(71) Applicant: THEAPRIN PHARMACEUTICALS, INC., Hauppauge, NY (US)

(72) Inventors: Philip V. FELICE, Smithtown, NY (US); Harry G. BRITTAIN, Milford, NJ (US)

(73) Assignee: THEAPRIN PHARMACEUTICALS, INC., Hauppauge, NY (US)

(21) Appl. No.: 16/617,880

(22) PCT Filed: May 30, 2018
(86) PCT No.: PCT/US2018/035127

§ 371 Date: Nov. 27, 2019

Related U.S. Application Data
(60) Provisional application No. 62/512,316, filed on May 30, 2017.

(51) Int. Cl. A61K 9/00; A61K 31/616; A61K 9/14; A61K 9/20

(52) U.S. Cl. CPC A61K 9/006; A61K 31/616; A61K 9/2009; A61K 9/143; A61K 9/2013; A61K 9/145

(57) ABSTRACT

A water-soluble aspirin, citric acid, sodium bicarbonate, L-theanine cocrystal composition which includes a quantity of acetylsalicylic acid is described. The composition may be created by a method including various steps, including a cocrystallization step. The water-soluble cocrystal composition is suitable for sublingual administration, preferably to humans.

### FIELD OF THE INVENTION

The invention relates to a novel sublingual formulation using a water-soluble cocrystal product of acetylsalicylic acid with citric acid, sodium bicarbonate, and L-theanine for the treatment of acute myocardial infarction.

### BACKGROUND OF THE INVENTION

Heart disease remains the leading cause of death in the US. About 790,000 people in the US have heart attacks each year. Of those, about 114,000 will die. The estimated annual incidence of heart attack in the US is 580,000 new attacks and 210,000 recurrent attacks. The average age at the first heart attack is 65.3 years for males and 71.8 years for females. Approximately every 40 seconds, an American will have a heart attack. The majority of Out of Hospital Cardiac Arrests (OHCA) occur at a home or residence (70%), public settings (19.8%) and Nursing Homes (10.6%) were the second and third most common locations of OHCA. Heart Attacks ($11.5 billion) and Coronary Heart Disease ($10.4 billion) were 2 of the 10 most expensive hospital principal discharge diagnoses.

### SUMMARY OF THE INVENTION

In one embodiment, the invention relates to a composition comprising aspirin with citric acid, sodium bicarbonate, and L-theanine. In an embodiment, the invention relates to a method of manufacturing a composition comprising aspirin with citric acid, sodium bicarbonate, and L-theanine. In some embodiments, the invention relates to a cocrystal aspirin, citric acid, sodium bicarbonate, L-theanine sublingual formulation, wherein the formulation can bypass the hepatic first pass effect, with enhanced dissolution rate and bioavailability when a rapid onset of action is desired, compared to the oral route of conventional aspirin. In one embodiment, the invention relates to a cocrystal aspirin, citric acid, sodium bicarbonate, L-theanine sublingual formulation that is expected to significantly reduce the median platelet aggregation inhibition time compared to the oral route of conventional aspirin. In one embodiment, the invention relates to a water- soluble cocrystal product of acetylsalicylic acid with citric acid, sodium bicarbonate, and L-theanine administered sublingually for the treatment of acute myocardial infarction.

In some embodiments, the invention relates to cocrystal compositions of a drug and the enantiomers, L- and D-isomers, D, L-racemic mixture, S- and R-isomers, S, R-racemic mixtures, all rotamers, tautomers, salt forms, and hydrates of the alpha and beta amino acids of theanine in which the N-substituted functional R₁-group [C₄ or gamma-CH₂-C(O)—NR₁] may contain linear, cyclic, or branched alkyl groups and derivatives thereof; linear, cyclic or branched alkenyl groups and derivatives thereof; and aromatic radicals (which may be aryl radicals) and derivatives thereof making up all the analogue forms of theanine.

In some embodiments the invention relates to one or more of the following pharmaceutical preparations:

### Preparation #4

| | |
|---|---|
| Aspirin | 379 mg (27.5%) |
| Theanine | 383 mg (27.8%) |
| NaHCO₃ | 269 mg (19.5%) |
| Citric Acid | 134 mg (9.7%) |
| PVP (K-25) | 69 mg (5.0%) |
| Mannitol (Pearlitol 200) | 135 mg (9.8%) |
| Mg Stearate | 7 mg (0.5%) |

### Preparation #5

| | |
|---|---|
| Aspirin | 376 mg (25.0%) |
| Theanine | 378 mg (25.2%) |
| NaHCO₃ | 261 mg (17.4%) |
| Citric Acid | 136 mg (9.1%) |
| PVP (K-25) | 74 mg (4.9%) |
| Mannitol (Pearlitol 200) | 269 mg (17.9%) |
| Mg Stearate | 7.5 mg (0.5%) |

### Preparation #6

| | |
|---|---|
| Aspirin | 376 mg (27.4.0%) |
| Theanine | 379 mg (27.7%) |
| NaHCO₃ | 266 mg (19.4%) |
| Citric Acid | 131 mg (9.6%) |
| PVP (K-25) | 66 mg (4.8%) |
| Mannitol (Pearlitol 200) | 102 mg (7.4%) |
| CP (Kollidon CL) | 43 mg (3.1%) |
| Mg Stearate | 7 mg (0.5%) |

### Preparation #7

| | |
|---|---|
| Aspirin | 381 mg (24.4%) |
| Theanine | 383 mg (24.6%) |
| NaHCO₃ | 264 mg (16.9%) |
| Citric Acid | 137 mg (8.8%) |
| PVP (K-25) | 77 mg (4.9%) |
| Mannitol (Pearlitol 200) | 258 mg (16.5%) |
| CP (Kollidon CL) | 52 mg (3.3%) |
| Mg Stearate | 8 mg (0.5%) |

The disintegration time for all preparations was less than 1 minute.

In one embodiment, the disclosure relates to a composition including aspirin, citric acid, sodium bicarbonate, and L-theanine. In some embodiments, the wt.% of aspirin in the composition is between about 20% and about 30%. In some embodiments, the wt.% of aspirin in the composition is selected from the group consisting of about 24.4%, about 25.0%, about 27.4%, and about 27.5%. In some embodiments, the wt.% of L-theanine in the composition is between about 20% and about 30%. In some embodiments, the wt.% of L-theanine in the composition is selected from the group consisting of about 24.6%, about 25.2%, about 27.7%, and about 27.8%. In some embodiments, the wt.% of sodium bicarbonate in the composition is between about 15% and about 25%. In some embodiments, the wt.% of sodium bicarbonate in the composition is selected from the group consisting of about 16.9%, about 17.4%, about 19.4%, and about 19.5%. In some embodiments, the wt.% of citric acid in the composition is between about 5% and about 15%. In some embodiments, the wt.% of citric acid in the composition is selected from the group consisting of about 8.8%, about 9.1%, about 9.6%, and about 9.7%. In some embodiments, the composition comprises one or more of a binder, an emulsifier, and a disintegrant. In some embodiments, the composition further includes polyvinylpyrrolidone at a wt.% of between about 2.5% and about 7.5%. In some embodiments, the wt.% of polyvinylpyrrolidone in the composition is selected from the group consisting of about 4.8%, about 4.9%, and about 5.0%. In some embodiments, the polyvinylpyrrolidone is cross-linked. In some embodiments, the wt.% of cross-linked polyvinylpyrrolidone in the composition is selected from the group consisting of about 3.1% and about 3.3%. In some embodiments, the composition further includes a sugar alcohol. In some embodiments, the composition further includes mannitol at a wt.% of between about 5% and about 20.0%. In some embodiments, the wt.% of mannitol in the composition is selected from the group consisting of about 7.4%, about 9.8%, about 16.5%, and about 17.9%. In some embodiments, the composition further includes a lubricant. In some embodiments, the composition further includes magnesium stearate at a wt.% of between about 0.01% and about 2.0%. In some embodiments, the wt.% of magnesium stearate in the composition is about 0.5%.

In one embodiment, the disclosure relates to a dosage form including aspirin, citric acid, sodium bicarbonate, and L-theanine. In some embodiments, the amount of aspirin in the dosage form is between about 300 mg and about 450 mg. In some embodiments, the amount of aspirin in the dosage form is selected from the group consisting of about 376 mg, about 379 mg, and about 381 mg. In some embodiments, the amount of L-theanine in the dosage form is between about 300 mg and about 450 mg. In some embodiments, the amount of L-theanine in the dosage form is selected from the group consisting of about 378 mg, about 379 mg, and about 383 mg. In some embodiments, the amount of sodium bicarbonate in the dosage form is between about 200 mg and about 350 mg. In some embodiments, the amount of sodium bicarbonate in the dosage form is selected from the group consisting of about 261 mg, about 264 mg, about 266 mg, and about 269 mg. In some embodiments, the amount of citric acid in the dosage form is between about 75 mg and about 200 mg. In some embodiments, the amount of citric acid in the dosage form is selected from the group consisting of about 131 mg, about 134 mg, about 136 mg, and about 137 mg. In some embodiments, the dosage form includes one or more of a binder, an emulsifier, and a disintegrant. In some embodiments, the dosage form further includes an amount of polyvinylpyrrolidone between about 50 mg and about 100 mg. In some embodiments, the amount of polyvinylpyrrolidone in the dosage form is selected from the group consisting of about 66 mg, about 69 mg, about 74 mg, and about 77 mg. In some embodiments, the polyvinylpyrrolidone is cross-linked. In some embodiments, the amount of cross-linked polyvinylpyrrolidone in the dosage form is between about 25 mg and about 75 mg. In some embodiments, the amount of cross-linked polyvinylpyrrolidone in the dosage form is selected from the group consisting of about 43 mg and about 52 mg. In some embodiments, the dosage form further includes a sugar alcohol. In some embodiments, the dosage form further includes an amount of mannitol between about 50 mg and about 300 mg. In some embodiments, the amount of mannitol in the dosage form is selected from the group consisting of about 102 mg, about 135 mg, about 258 mg, and about 269 mg. In some embodiments, the dosage form further includes a lubricant. In some embodiments, the dosage form further includes an amount of magnesium stearate of between about 2.5 mg and about 15 mg. In some embodiments, the amount of magnesium stearate in the dosage form is selected from the group consisting of about 7.0 mg, about 7.5 mg, and about 8 mg.

In some embodiments, the invention relates to a method of treating acute myocardial infarction in a subject in need thereof, the method including administering to the subject a sublingual cocrystal aspirin with citric acid, sodium bicarbonate, and L-theanine. In one embodiment, the sublingual aspirin, citric acid, sodium bicarbonate, L-theanine cocrystal are directly absorbed in the subject's bloodstream and bypass the hepatic first pass effect.

### DETAILED DESCRIPTION

Early administration of a novel sublingual aspirin, citric acid, sodium bicarbonate, L-theanine cocrystal formulation for the treatment of acute myocardial infarction is paramount and could start benefiting the patient in a matter of minutes, whereas the full benefit of traditional aspirin may not take effect until major sequelae, like ventricular tachycardia, ventricular fibrillation, complete heart block, or death has occurred. As such, there is a clear unmet need for a novel sublingual aspirin, citric acid, sodium bicarbonate, L-theanine cocrystal formulation with improved pharmacokinetics and pharmacodynamics in patients presenting with acute myocardial infarction. The present invention satisfies these and other medical needs and overcomes deficiencies found in the prior art.

The advantages of an aspirin, citric acid, sodium bicarbonate, L-theanine cocrystal sublingual formulation compared to the oral route of conventional aspirin includes enhanced dissolution rate and bioavailability when a rapid onset of action is desired. The vascular network of blood vessels under the tongue provides for an increased absorption of the drug compared to the oral route. As such, the sublingual network of blood vessels result in a faster dissolution rate, especially if the pH of the saliva is greater than 6. The sublingual route bypasses the hepatic first pass effect resulting in increased bioavailability of the drug since the drug is absorbed directly into the systemic circulation instead of being absorbed through the gastrointestinal tract where it is first delivered to the liver by the portal vein then metabolized in the liver prior to entering the systemic circulation. Patients with a history of acute coronary syndrome can self-administer the cocrystal aspirin, citric acid, sodium bicarbonate, L-theanine sublingual formulation during an acute onset of chest pain while at home or work prior to the ambulance arriving, while in the wilderness, or during air travel. Patients with a history of dysphagia (difficulty swallowing), or a history of odynophagia (pain on swallowing), would benefit from the sublingual route.

One significant disadvantage of the sublingual route occurs in patients who smoke. Smokers will not fully benefit from the sublingual route since smoking causes vasoconstriction of the blood vessels, which will decrease the absorption of the medication.

The ongoing interest in modification of drug substances whose physical properties are less than desirable has led to significant study of issues associated with polymorphism and solvatomorphism. More recently, it has been recognized that many substances may cocrystallize in a single continuous lattice structure, leading pharmaceutical scientists into new areas of crystal engineering.

Cocrystals are mixed crystals where the cocrystal is a structurally homogeneous crystalline material that has been formed from discrete neutral molecular species that are solids at ambient temperatures. Cocrystals are characterized by two or more molecules that associate but do not bond on the molecular level.

Cocrystals represent novel forms of drug substances that would be suitable for incorporation in pharmaceutical solid dosage forms, and should enable formulation scientists to overcome a variety of problems that are encountered during development of traditional formulations. One could consider cocrystals as being an alternative to polymorphs, solvatomorphs, and salts, as cocrystals represent a different approach to solve problems related to dissolution, crystallinity, and hygroscopicity, for example.

Cocrystals are attractive to the pharmaceutical industry because they offer opportunities to modify the chemical and/or physical properties of an API without the need to make or break covalent bonds.

Unfortunately, it is not yet possible to predict whether two substances will cocrystallize or not, and therefore cocrystal screening studies are largely empirical in nature.

Given the structural similarity of glutamine and glutamic acid with theanine, and the fact that neither glutamine nor glutamic acid forms a cocrystal with aspirin, one might have been led to deduce that theanine would not form a cocrystal with aspirin either. In spite of this expectation, aspirin does form a cocrystal with theanine, but aspirin does not form a cocrystal with either glutamine or glutamic acid.

Conformers depicted below are highly structurally related to L-theanine:

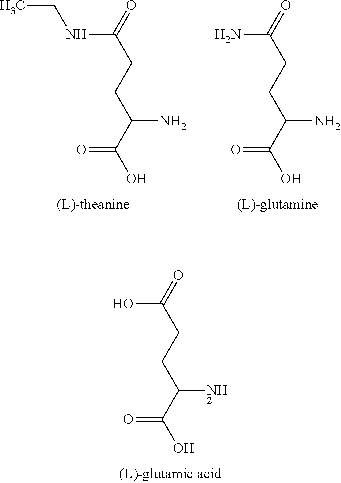

Cocrystal engineering may be used to improve one or more physical properties such as solubility, stability, and dissolution rate of the active pharmaceutical ingredient of selected treatment or prevention.

Salicylic acid is the active metabolite of aspirin.

Therapeutic compounds, such as aspirin, are most stable in a crystalline form, but can display poor aqueous solubility and slow dissolution rates. These properties have the tendency reduce the bioavailability of the active pharmaceutical ingredient (API), thereby slowing absorption.

Myocardial infarction is most often caused by rupture of an atherosclerotic lesion in a coronary artery. This rupture causes the formation of a coronary thrombus or a blood clot that occludes the artery, preventing the artery from supplying blood and oxygen to the myocardium normally supplied by that vessel. As a result, ischemic death of cardiomyocytes ensues.

Activated macrophages and T-lymphocytes localized at the site of plaque rupture releases metalloproteases and cytokines which weaken the fibrosis cap, rendering it susceptible to erosion or tearing due to the shear stress exerted by the blood flow. Plaque rupture exposes subendothelial collagen, which serves as a site of platelet adhesion, activation and aggregation. This results in the release of substances such as thromoboxane A2, fibrinogen, 5-hydroxytryptamine (serotonin), platelet activation factor and ADP (adenosine diphosphate), which further promotes platelet aggregation. Activation of the clotting cascade, leads to fibrin formation and propagation of the occlusive thrombus. As the thrombus builds upon the ruptured plaque it ultimately occludes the artery. Atherosclerotic plaque rupture with superimposed thrombus accounts for 95% of the cases of acute myocardial infarctions.

Inflammation and inflammatory cell infiltration are the hallmarks of myocardial infarction and reperfusion injury. Ischemic cardiac injury activates the innate immune response via toll-like receptors and upregulates chemokine and cytokine expressions in the infarcted heart. Sequential infiltration of the injured myocardium with neutrophils, monocytes and their descendant macrophages, dendritic cells, and lymphocytes contributes to the initiation and resolution of inflammation, infarct healing, angiogenesis, and ventricular remodeling. Both detrimental effects and a beneficial role in the pathophysiology of myocardial infarction (MI) and reperfusion injury may be attributed to the subset heterogeneity and functional diversity of these inflammatory cells.

Aspirin irreversibly inhibits platelet cyclooxygenase 1 (COX-1) through acetylation of the amino acid serine at position 529, thereby preventing arachidonic acid access to the COX-1 catalytic site through steric hindrance. By inhibiting COX-1, the platelet is unable to synthesize prostaglandin H2, which would otherwise be converted to thromboxane A2, which causes platelet aggregation, an early step in the coagulation cascade.

Theanine is found in green tea leaves Camellia sinensis and in the non-edible mushroom Xerocomus badius, but is otherwise rare in nature. Tea is the second most consumed beverage in the world. Monks have been drinking tea containing theanine for over 4,000 years. L-theanine is an ingredient in fruit juices and drinks, non-herbal teas, sport beverages, bottled waters, chocolate bars, hard candies, breath mints, and chewing gum. Theanine is extremely safe, with a LD50 toxicity of >5000 mg/kg in rats. According to the FDA's GRAS assessment of L-Theanine that was based on statistical analysis of potential dietary intake, it was estimated that the mean theanine consumption would be 682 mg/person/day, and the 90^{th} percentile consumption would be 1284 mg (1.28 g)/person/day.

L-theanine (N-ethyl-L-glutamine) an amino acid analog of glutamine, is a water-soluble, non-protein amino acid. It is an odorless, white crystalline powder that is soluble in water and transparent in solution. L-theanine has a Chemical Abstracts Service (CAS) Registry Number of 3081-61-6 and a GRAS classification (GRAS Notice Number: GRN 000209). L-theanine has the empirical formula C₇H₁₄N₂O₃, molecular weight of 174.20, pKa of 2.35, and a melting point of 217-218° C. Theanine is hydrolyzed in the kidney to glutamic acid and ethylamine by the enzyme glutaminase.

### Chemical Structure of L-Theanine

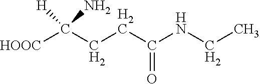

Theanine is known to have several mechanisms of action. Thrombin is a serine protease which enables the conversion of fibrinogen into fibrin during the coagulation cascade resulting in clot formation. Theanine by itself, is a potent inhibitor of thrombin stimulated thromboxane formulation in whole blood. As such, theanine has an anticoagulant effect. This anticoagulant effect of theanine could be beneficial for the treatment of acute myocardial infarction when administering cocrystals of aspirin with citric acid, sodium bicarbonate, and L-theanine sublingually. Theanine promotes alpha wave production in the brain, causing a relaxed mental and physical state without causing drowsiness. This relaxed mental and physical state would be beneficial in a patient presenting with an acute myocardial infarction.

Theanine enhances the solubility of aspirin in water. Being 5-N-ethyl glutamine, theanine differs from glutamine by the CH₂-CH₃ (ethyl) group replacing hydrogen. The N-ethyl group confers on theanine its active properties. Theanine and its analogues form zwitterions at neutral pH. The ion charges are available to pair through the cationic or protonated alpha amino group with the ortho carboxylate anion of acetylsalicylic acid. This can be further stabilized by hydrogen bonding to the ethylcarboxamide's nitrogen (this ethylamido group is the functional signature of theanine type molecules). Crystals of this ion pair may encapsulate more water molecules than either molecule alone. This gives the complex its easy solvation on dissolution in water or buffer.

Aspirin protects against several kinds of toxicity, including excitotoxicity (glutamate), dopamine toxicity, and oxidative free radical toxicity.

Excitotoxicity is the pathological process responsible for neuronal cell death due to excessive stimulation by neurotransmitters such as glutamate. Pathologically high levels of glutamate, can cause excitotoxicity by allowing high levels of calcium ions (Ca²⁺) to enter the cell. This calcium influx (Ca²⁺) into the cell results in liberation of a number of enzymes resulting in neuronal death. Theanine has been shown to bind to AMPA (α-amino-3-hydroxy-5-methyl-4-isoxazolepropionic acid), Kainate, NMDA (N-methyl-D-aspartate) and group 1 mGlu receptors, thereby opposing excitatory neurotransmitters.

GABA (Gamma-Aminobutyric Acid) is the most widespread inhibitory neurotransmitter of the brain. When GABA levels are decreased there is an augmentation of nerve impulses in the neuron. Theanine increases GABA levels in the brain, opposing excess stimulation of nerve impulses by excitatory neurotransmitters such as glutamate, which can be found in patients with stroke and traumatic brain injury.

L-theanine, crosses the blood-brain barrier via the large neutral amino acid leucine-preferring transport system, and has been demonstrated to produce significant increases in serotonin and/or dopamine concentrations in the brain principally in the striatum, hypothalamus and hippocampus. L-Theanine's ability to increase serotonin and dopamine concentration in the brain would be beneficial in the treatment of migraine patients where the serotonin and dopamine levels are usually decreased.

A novel sublingual water soluble cocrystal aspirin with citric acid, sodium bicarbonate, and L-theanine that crosses the blood brain barrier would be neuroprotective against a variety of central nervous system disorders associated with glutamate excitotoxicity such as, spinal cord injury, stroke, traumatic brain injury, multiple sclerosis, Alzheimer's disease, Parkinson's disease, Huntington's disease, and amyotrophic lateral sclerosis.

Researchers at the Boyce Thompson Institute and John Hopkins University discovered that salicylic acid, the active metabolite of aspirin binds to binds to Glyceraldehyde 3-Phosphate Dehydrogenase (GAPDH) preventing the enzyme from entering the nucleus of the neurons where it enhances protein turnover leading to cell death. GAPDH is believed to play a major role in neurodegenerative diseases, including Alzheimer's, Parkinson's, and Huntington's diseases. A novel sublingual water soluble cocrystal aspirin with citric acid, sodium bicarbonate, and L-theanine that crosses the blood brain barrier would be paramount in preventing neuronal cell death by binding salicylic acid to Glyceraldehyde 3-Phosphate Dehydrogenase.

Theanine inhibits the efflux of chemotherapeutic agents such as, doxorubicin, idarubicin, cisplatin, and irinotecan resulting in an elevated level of the drugs within cancer cells which strongly inhibits the tumor. Theanine blocks the export of doxorubicin, idarubicin, cisplatin, and irinotecan from cancer cells by blocking the glutamate and glutathione transporter mechanisms. Cancer cells use glutathione to detoxify doxorubicin, idarubicin, cisplatin, and irinotecan and escort the drugs out of cells. Theanine is able to interfere with this process due to its structural similarity to glutamate.

The various features of novelty which characterize the invention are pointed out with particularity in the claims annexed to and forming a part of this disclosure. For a better understanding of the invention, its operation, advantages, and specific objects attained by its uses, reference is made to the descriptive matter in which a preferred embodiment of the invention is illustrated.

Accordingly, it is an object of the invention to provide a method utilizing a cocrystal composition of aspirin with citric acid, sodium bicarbonate, and L-theanine which is readily administered to humans via the sublingual route, bypassing the hepatic first pass effect, enhancing dissolution rate and bioavailability.

Another object of the invention is to provide a water-soluble aspirin, citric acid, sodium bicarbonate, theanine cocrystal composition having the above characteristics and which is rapidly water soluble.

Another object of the invention is to provide a water-soluble aspirin, citric acid, sodium bicarbonate, theanine cocrystal formulation suitable for sublingual administration having the above characteristics and which allow for rapid delivery of acetylsalicylic acid into the blood stream.

Still another object of the invention is to provide a water-soluble aspirin, citric acid, sodium bicarbonate, theanine cocrystal formulation suitable for sublingual administration having the above characteristics and which allow for rapid delivery of therapeutic quantities of theanine into the blood stream.

A further object of the invention is to provide method of administering a water-soluble aspirin, citric acid, sodium bicarbonate, theanine cocrystal composition sublingually in humans that provides enhanced dissolution and bioavailability and is suitable for treatment of acute myocardial infarction.

Still a further object of the invention is to provide a water-soluble aspirin, citric acid, sodium bicarbonate, theanine cocrystal composition suitable for sublingual administration having the above characteristics and which is expected to reduce the median platelet aggregation inhibition time compared to the oral route of conventional aspirin.

Yet a further object of the invention is to provide a water-soluble aspirin, citric acid, sodium bicarbonate, theanine cocrystal formulation suitable for sublingual administration having the above characteristics and which bypasses the gastrointestinal tract without causing the gastrointestinal irritation, erosions, or bleeding which may occur with conventional oral aspirin use.

Embodiments of the invention may include cocrystal compositions of drugs from the classes listed below and the enantiomers, L- and D-isomers, D, L-racemic mixture, S- and R-isomers, S, R-racemic mixtures, all rotamers, tautomers, salt forms, and hydrates of the alpha and beta amino acids of theanine in which the N-substituted functional R1-group [C₄ or gamma-CH2-C(O)—NR₁] may contain linear, cyclic, or branched alkyl groups and derivatives thereof; linear, cyclic or branched alkenyl groups and derivatives thereof; and aromatic radicals (which may be aryl radicals) and derivatives thereof making up all the analogue forms of theanine.

In some embodiments of the invention, the theanine enantiomer further comprises a carbohydrate functional group thereon. In these embodiments, the carbohydrate functional group may be of the L-configuration or the D-configuration. In these embodiments, the carbohydrates employed may be monosaccharides, disaccharides, trisaccharides, oligosaccharides or polysaccharides.

In some embodiments of the invention, the theanine enantiomer further comprises an amino acid functional group thereon. In certain of these embodiments, the amino acid functional group is a dipeptide.

Sodium bicarbonate is a white crystalline powder that is very water soluble, formula NaHCO₃, CAS number 144-55-8, molecular weight of 84.006 g/mol., pKa's of 6.4 and 10.3. Without being limited to a specific theory of the invention, sodium bicarbonate (NaHCO₃) deprotonates the carboxylic acid of aspirin to produce a sodium acetylsalicylate salt which is more soluble in water due to the presence of a charged carboxylate group. Aspirin hydrolysis is pH independent between pH 5-8, meaning in the range up to saturation, bicarbonate is a specific base catalyst for generating the aspirin anion. Aspirin hydrolysis of the acetate is pH independent and solely dependent on the concentration of the aspirin carboxylate anion. At molar equivalents greater than 1.2, proton abstraction from the acid of aspirin is super-fast and complete.

Citric Acid, 2-hydroxypropane-1,2,3-tricarboxylic acid, CAS Number 77-92-9, formula C₆H₈O₇, with a molecular weight of 192.12 g/mol. is very soluble in water. It is tribasic with pKa 1-3 of 5.21, 4.28, and 2.92. Under physiological conditions one of the acid groups is deprotonated allowing the conjugate base of the acid to bind other molecules. Citric acid being acidic dissolves calcium ion based coagulants by chelating calcium out of clot complexes in the blood. As such, the salt of citric acid has an anticoagulant effect due to its calcium chelating ability. This anticoagulant effect would be beneficial in patients suffering from acute myocardial infarction when administered as a sublingual aspirin, citric acid, sodium bicarbonate, L-theanine cocrystal. The citric acid in the formulation reacts with sodium bicarbonate resulting in a buffer, disintegrant and favorable compressability.

In one embodiment, the disclosure relates to a composition including aspirin, citric acid, sodium bicarbonate, and L-theanine. In some embodiments, the wt.% of aspirin in the composition is between about 20% and about 30%. In some embodiments, the wt.% of aspirin in the composition is selected from the group consisting of about 20.00%, about 20.10%, about 20.20%, about 20.30%, about 20.40%, about 20.50%, about 20.60%, about 20.70%, about 20.80%, about 20.90%, about 21.00%, about 21.10%, about 21.20%, about 21.30%, about 21.40%, about 21.50%, about 21.60%, about 21.70%, about 21.80%, about 21.90%, about 22.00%, about 22.10%, about 22.20%, about 22.30%, about 22.40%, about 22.50%, about 22.60%, about 22.70%, about 22.80%, about 22.90%, about 23.00%, about 23.10%, about 23.20%, about 23.30%, about 23.40%, about 23.50%, about 23.60%, about 23.70%, about 23.80%, about 23.90%, about 24.00%, about 24.10%, about 24.20%, about 24.30%, about 24.40%, about 24.50%, about 24.60%, about 24.70%, about 24.80%, about 24.90%, about 25.00%, about 25.10%, about 25.20%, about 25.30%, about 25.40%, about 25.50%, about 25.60%, about 25.70%, about 25.80%, about 25.90%, about 26.00%, about 26.10%, about 26.20%, about 26.30%, about 26.40%, about 26.50%, about 26.60%, about 26.70%, about 26.80%, about 26.90%, about 27.00%, about 27.10%, about 27.20%, about 27.30%, about 27.40%, about 27.50%, about 27.60%, about 27.70%, about 27.80%, about 27.90%, about 28.00%, about 28.10%, about 28.20%, about 28.30%, about 28.40%, about 28.50%, about 28.60%, about 28.70%, about 28.80%, about 28.90%, about 29.00%, about 29.10%, about 29.20%, about 29.30%, about 29.40%, about 29.50%, about 29.60%, about 29.70%, about 29.80%, about 29.90%, and about 30.00%.

In some embodiments, the wt.% of L-theanine in the composition is between about 20% and about 30%. In some embodiments, the wt.% of L-theanine in the composition is selected from the group consisting of about 20.00%, about 20.10%, about 20.20%, about 20.30%, about 20.40%, about 20.50%, about 20.60%, about 20.70%, about 20.80%, about 20.90%, about 21.00%, about 21.10%, about 21.20%, about 21.30%, about 21.40%, about 21.50%, about 21.60%, about 21.70%, about 21.80%, about 21.90%, about 22.00%, about 22.10%, about 22.20%, about 22.30%, about 22.40%, about 22.50%, about 22.60%, about 22.70%, about 22.80%, about 22.90%, about 23.00%, about 23.10%, about 23.20%, about 23.30%, about 23.40%, about 23.50%, about 23.60%, about 23.70%, about 23.80%, about 23.90%, about 24.00%, about 24.10%, about 24.20%, about 24.30%, about 24.40%, about 24.50%, about 24.60%, about 24.70%, about 24.80%, about 24.90%, about 25.00%, about 25.10%, about 25.20%, about 25.30%, about 25.40%, about 25.50%, about 25.60%, about 25.70%, about 25.80%, about 25.90%, about 26.00%, about 26.10%, about 26.20%, about 26.30%, about 26.40%, about 26.50%, about 26.60%, about 26.70%, about 26.80%, about 26.90%, about 27.00%, about 27.10%, about 27.20%, about 27.30%, about 27.40%, about 27.50%, about 27.60%, about 27.70%, about 27.80%, about 27.90%, about 28.00%, about 28.10%, about 28.20%, about 28.30%, about 28.40%, about 28.50%, about 28.60%, about 28.70%, about 28.80%, about 28.90%, about 29.00%, about 29.10%, about 29.20%, about 29.30%, about 29.40%, about 29.50%, about 29.60%, about 29.70%, about 29.80%, about 29.90%, and about 30.00%.

In some embodiments, the wt.% of sodium bicarbonate in the composition is between about 15% and about 25%. In some embodiments, the wt.% of sodium bicarbonate in the composition is selected from the group consisting of about 15.00%, about 15.10%, about 15.20%, about 15.30%, about 15.40%, about 15.50%, about 15.60%, about 15.70%, about 15.80%, about 15.90%, about 16.00%, about 16.10%, about 16.20%, about 16.30%, about 16.40%, about 16.50%, about 16.60%, about 16.70%, about 16.80%, about 16.90%, about 17.00%, about 17.10%, about 17.20%, about 17.30%, about 17.40%, about 17.50%, about 17.60%, about 17.70%, about 17.80%, about 17.90%, about 18.00%, about 18.10%, about 18.20%, about 18.30%, about 18.40%, about 18.50%, about 18.60%, about 18.70%, about 18.80%, about 18.90%, about 19.00%, about 19.10%, about 19.20%, about 19.30%, about 19.40%, about 19.50%, about 19.60%, about 19.70%, about 19.80%, about 19.90%, about 20.00%, about 20.10%, about 20.20%, about 20.30%, about 20.40%, about 20.50%, about 20.60%, about 20.70%, about 20.80%, about 20.90%, about 21.00%, about 21.10%, about 21.20%, about 21.30%, about 21.40%, about 21.50%, about 21.60%, about 21.70%, about 21.80%, about 21.90%, about 22.00%, about 22.10%, about 22.20%, about 22.30%, about 22.40%, about 22.50%, about 22.60%, about 22.70%, about 22.80%, about 22.90%, about 23.00%, about 23.10%, about 23.20%, about 23.30%, about 23.40%, about 23.50%, about 23.60%, about 23.70%, about 23.80%, about 23.90%, about 24.00%, about 24.10%, about 24.20%, about 24.30%, about 24.40%, about 24.50%, about 24.60%, about 24.70%, about 24.80%, about 24.90%, and about 25.00%.

In some embodiments, the wt.% of citric acid in the composition is between about 5% and about 15%. In some embodiments, the wt.% of citric acid in the composition is selected from the group consisting of about 5.00%, about 5.10%, about 5.20%, about 5.30%, about 5.40%, about 5.50%, about 5.60%, about 5.70%, about 5.80%, about 5.90%, about 6.00%, about 6.10%, about 6.20%, about 6.30%, about 6.40%, about 6.50%, about 6.60%, about 6.70%, about 6.80%, about 6.90%, about 7.00%, about 7.10%, about 7.20%, about 7.30%, about 7.40%, about 7.50%, about 7.60%, about 7.70%, about 7.80%, about 7.90%, about 8.00%, about 8.10%, about 8.20%, about 8.30%, about 8.40%, about 8.50%, about 8.60%, about 8.70%, about 8.80%, about 8.90%, about 9.00%, about 9.10%, about 9.20%, about 9.30%, about 9.40%, about 9.50%, about 9.60%, about 9.70%, about 9.80%, about 9.90%, about 10.00%, about 10.10%, about 10.20%, about 10.30%, about 10.40%, about 10.50%, about 10.60%, about 10.70%, about 10.80%, about 10.90%, about 11.00%, about 11.10%, about 11.20%, about 11.30%, about 11.40%, about 11.50%, about 11.60%, about 11.70%, about 11.80%, about 11.90%, about 12.00%, about 12.10%, about 12.20%, about 12.30%, about 12.40%, about 12.50%, about 12.60%, about 12.70%, about 12.80%, about 12.90%, about 13.00%, about 13.10%, about 13.20%, about 13.30%, about 13.40%, about 13.50%, about 13.60%, about 13.70%, about 13.80%, about 13.90%, about 14.00%, about 14.10%, about 14.20%, about 14.30%, about 14.40%, about 14.50%, about 14.60%, about 14.70%, about 14.80%, about 14.90%, and about 15.00%.

In some embodiments, the composition comprises one or more of a binder, an emulsifier, and a disintegrant. In some embodiments, the composition further includes polyvinylpyrrolidone at a wt.% of between about 2.5% and about 7.5%. In some embodiments, the wt.% of polyvinylpyrrolidone in the composition is selected from the group consisting of some embodiments, the wt.% of citric acid in the composition is selected from the group consisting of about 2.50%, about 2.60%, about 2.70%, about 2.80%, about 2.90%, about 3.00%, about 3.10%, about 3.20%, about 3.30%, about 3.40%, about 3.50%, about 3.60%, about 3.70%, about 3.80%, about 3.90%, about 4.00%, about 4.10%, about 4.20%, about 4.30%, about 4.40%, about 4.50%, about 4.60%, about 4.70%, about 4.80%, about 4.90%, about 5.00%, about 5.10%, about 5.20%, about 5.30%, about 5.40%, about 5.50%, about 5.60%, about 5.70%, about 5.80%, about 5.90%, about 6.00%, about 6.10%, about 6.20%, about 6.30%, about 6.40%, about 6.50%, about 6.60%, about 6.70%, about 6.80%, about 6.90%, about 7.00%, about 7.10%, about 7.20%, about 7.30%, about 7.40%, and about 7.50%.

In some embodiments, the polyvinylpyrrolidone is cross-linked. In some embodiments, the wt.% of cross-linked polyvinylpyrrolidone in the composition is selected from the group consisting of about 2.50%, about 2.60%, about 2.70%, about 2.80%, about 2.90%, about 3.00%, about 3.10%, about 3.20%, about 3.30%, about 3.40%, about 3.50%, about 3.60%, about 3.70%, about 3.80%, about 3.90%, and about 4.00%.

In some embodiments, the composition further includes a sugar alcohol. In some embodiments, the composition further includes mannitol at a wt.% of between about 5% and about 20.0%. In some embodiments, the wt.% of mannitol in the composition is selected from the group consisting of about 5.00%, about 5.10%, about 5.20%, about 5.30%, about 5.40%, about 5.50%, about 5.60%, about 5.70%, about 5.80%, about 5.90%, about 6.00%, about 6.10%, about 6.20%, about 6.30%, about 6.40%, about 6.50%, about 6.60%, about 6.70%, about 6.80%, about 6.90%, about 7.00%, about 7.10%, about 7.20%, about 7.30%, about 7.40%, about 7.50%, about 7.60%, about 7.70%, about 7.80%, about 7.90%, about 8.00%, about 8.10%, about 8.20%, about 8.30%, about 8.40%, about 8.50%, about 8.60%, about 8.70%, about 8.80%, about 8.90%, about 9.00%, about 9.10%, about 9.20%, about 9.30%, about 9.40%, about 9.50%, about 9.60%, about 9.70%, about 9.80%, about 9.90%, about 10.00%, about 10.10%, about 10.20%, about 10.30%, about 10.40%, about 10.50%, about 10.60%, about 10.70%, about 10.80%, about 10.90%, about 11.00%, about 11.10%, about 11.20%, about 11.30%, about 11.40%, about 11.50%, about 11.60%, about 11.70%, about 11.80%, about 11.90%, about 12.00%, about 12.10%, about 12.20%, about 12.30%, about 12.40%, about 12.50%, about 12.60%, about 12.70%, about 12.80%, about 12.90%, about 13.00%, about 13.10%, about 13.20%, about 13.30%, about 13.40%, about 13.50%, about 13.60%, about 13.70%, about 13.80%, about 13.90%, about 14.00%, about 14.10%, about 14.20%, about 14.30%, about 14.40%, about 14.50%, about 14.60%, about 14.70%, about 14.80%, about 14.90%, about 15.00%, about 15.10%, about 15.20%, about 15.30%, about 15.40%, about 15.50%, about 15.60%, about 15.70%, about 15.80%, about 15.90%, about 16.00%, about 16.10%, about 16.20%, about 16.30%, about 16.40%, about 16.50%, about 16.60%, about 16.70%, about 16.80%, about 16.90%, about 17.00%, about 17.10%, about 17.20%, about 17.30%, about 17.40%, about 17.50%, about 17.60%, about 17.70%, about 17.80%, about 17.90%, about 18.00%, about 18.10%, about 18.20%, about 18.30%, about 18.40%, about 18.50%, about 18.60%, about 18.70%, about 18.80%, about 18.90%, about 19.00%, about 19.10%, about 19.20%, about 19.30%, about 19.40%, about 19.50%, about 19.60%, about 19.70%, about 19.80%, about 19.90%, and about 20.00%.

In some embodiments, the composition further includes a lubricant. In some embodiments, the composition further includes magnesium stearate at a wt.% of between about 0.01% and about 2.0%. In some embodiments, the wt.% of magnesium stearate in the composition is about 0.01%, about 0.02%, about 0.03%, about 0.04%, about 0.05%, about 0.06%, about 0.07%, about 0.08%, about 0.09%, about 0.10%, about 0.11%, about 0.12%, about 0.13%, about 0.14%, about 0.15%, about 0.16%, about 0.17%, about 0.18%, about 0.19%, about 0.20%, about 0.21%, about 0.22%, about 0.23%, about 0.24%, about 0.25%, about 0.26%, about 0.27%, about 0.28%, about 0.29%, about 0.30%, about 0.31%, about 0.32%, about 0.33%, about 0.34%, about 0.35%, about 0.36%, about 0.37%, about 0.38%, about 0.39%, about 0.40%, about 0.41%, about 0.42%, about 0.43%, about 0.44%, about 0.45%, about 0.46%, about 0.47%, about 0.48%, about 0.49%, about 0.50%, about 0.51%, about 0.52%, about 0.53%, about 0.54%, about 0.55%, about 0.56%, about 0.57%, about 0.58%, about 0.59%, about 0.60%, about 0.61%, about 0.62%, about 0.63%, about 0.64%, about 0.65%, about 0.66%, about 0.67%, about 0.68%, about 0.69%, about 0.70%, about 0.71%, about 0.72%, about 0.73%, about 0.74%, about 0.75%, about 0.76%, about 0.77%, about 0.78%, about 0.79%, about 0.80%, about 0.81%, about 0.82%, about 0.83%, about 0.84%, about 0.85%, about 0.86%, about 0.87%, about 0.88%, about 0.89%, about 0.90%, about 0.91%, about 0.92%, about 0.93%, about 0.94%, about 0.95%, about 0.96%, about 0.97%, about 0.98%, about 0.99%, about 1.00%, about 1.01%, about 1.02%, about 1.03%, about 1.04%, about 1.05%, about 1.06%, about 1.07%, about 1.08%, about 1.09%, about 1.10%, about 1.11%, about 1.12%, about 1.13%, about 1.14%, about 1.15%, about 1.16%, about 1.17%, about 1.18%, about 1.19%, about 1.20%, about 1.21%, about 1.22%, about 1.23%, about 1.24%, about 1.25%, about 1.26%, about 1.27%, about 1.28%, about 1.29%, about 1.30%, about 1.31%, about 1.32%, about 1.33%, about 1.34%, about 1.35%, about 1.36%, about 1.37%, about 1.38%, about 1.39%, about 1.40%, about 1.41%, about 1.42%, about 1.43%, about 1.44%, about 1.45%, about 1.46%, about 1.47%, about 1.48%, about 1.49%, about 1.50%, about 1.51%, about 1.52%, about 1.53%, about 1.54%, about 1.55%, about 1.56%, about 1.57%, about 1.58%, about 1.59%, about 1.60%, about 1.61%, about 1.62%, about 1.63%, about 1.64%, about 1.65%, about 1.66%, about 1.67%, about 1.68%, about 1.69%, about 1.70%, about 1.71%, about 1.72%, about 1.73%, about 1.74%, about 1.75%, about 1.76%, about 1.77%, about 1.78%, about 1.79%, about 1.80%, about 1.81%, about 1.82%, about 1.83%, about 1.84%, about 1.85%, about 1.86%, about 1.87%, about 1.88%, about 1.89%, about 1.90%, about 1.91%, about 1.92%, about 1.93%, about 1.94%, about 1.95%, about 1.96%, about 1.97%, about 1.98%, about 1.99%, and about 2.00%.

In one embodiment, the disclosure relates to a dosage form including aspirin, citric acid, sodium bicarbonate, and L-theanine. In some embodiments, the amount of aspirin in the dosage form is between about 300 mg and about 450 mg. In some embodiments, the amount of aspirin in the dosage form is selected from the group consisting of about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 376 mg, about 379 mg, about 380 mg, about 381 mg, about 395 mg, about 390 mg, about 395 mg, about 400 mg, about 405 mg, about 410 mg, about 415 mg, about 420 mg, about 425 mg, about 430 mg, about 435 mg, about 440 mg, about 445 mg, and about 450 mg.

In some embodiments, the amount of L-theanine in the dosage form is between about 300 mg and about 450 mg. In some embodiments, the amount of L-theanine in the dosage form is selected from the group consisting of about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 376 mg, about 379 mg, about 380 mg, about 381 mg, about 395 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 378 mg, about 379 mg, and about 383 mg.

In some embodiments, the amount of sodium bicarbonate in the dosage form is between about 200 mg and about 350 mg. In some embodiments, the amount of sodium bicarbonate in the dosage form is selected from the group consisting of about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 261 mg, about 264 mg, about 266 mg, and about 269 mg.

In some embodiments, the amount of citric acid in the dosage form is between about 75 mg and about 200 mg. In some embodiments, the amount of citric acid in the dosage form is selected from the group consisting of about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 131 mg, about 134 mg, about 136 mg, and about 137 mg.

In some embodiments, the dosage form includes one or more of a binder, an emulsifier, and a disintegrant. In some embodiments, the dosage form further includes an amount of polyvinylpyrrolidone between about 50 mg and about 100 mg. In some embodiments, the amount of polyvinylpyrrolidone in the dosage form is selected from the group consisting of about 50 mg, about 55 mg, about 60 mg, about 65 mg, about 70 mg, about 75 mg, about 80 mg, about 85 mg, about 90 mg, about 95 mg, about 100 mg, about 66 mg, about 69 mg, about 74 mg, and about 77 mg.

In some embodiments, the polyvinylpyrrolidone is cross-linked. In some embodiments, the amount of cross-linked polyvinylpyrrolidone in the dosage form is between about 25 mg and about 75 mg. In some embodiments, the amount of cross-linked polyvinylpyrrolidone in the dosage form is selected from the group consisting of about 25 mg, about 30 mg, about 35 mg, about 40 mg, about 43 mg, about 45 mg, about 50 mg, about 52 mg, about 55 mg, about 60 mg, about 65 mg, about 70 mg, and about 75 mg.

In some embodiments, the dosage form further includes a sugar alcohol. In some embodiments, the dosage form further includes an amount of mannitol between about 50 mg and about 300 mg. In some embodiments, the amount of mannitol in the dosage form is selected from the group consisting of 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 102 mg, about 135 mg, about 258 mg, and about 269 mg.

In some embodiments, the dosage form further includes a lubricant. In some embodiments, the dosage form further includes an amount of magnesium stearate of between about 2.5 mg and about 15 mg. In some embodiments, the amount of magnesium stearate in the dosage form is selected from the group consisting of about 2.50 mg, about 2.55 mg, about 2.60 mg, about 2.65 mg, about 2.70 mg, about 2.75 mg, about 2.80 mg, about 2.85 mg, about 2.90 mg, about 2.95 mg, about 3.00 mg, about 3.05 mg, about 3.10 mg, about 3.15 mg, about 3.20 mg, about 3.25 mg, about 3.30 mg, about 3.35 mg, about 3.40 mg, about 3.45 mg, about 3.50 mg, about 3.55 mg, about 3.60 mg, about 3.65 mg, about 3.70 mg, about 3.75 mg, about 3.80 mg, about 3.85 mg, about 3.90 mg, about 3.95 mg, about 4.00 mg, about 4.05 mg, about 4.10 mg, about 4.15 mg, about 4.20 mg, about 4.25 mg, about 4.30 mg, about 4.35 mg, about 4.40 mg, about 4.45 mg, about 4.50 mg, about 4.55 mg, about 4.60 mg, about 4.65 mg, about 4.70 mg, about 4.75 mg, about 4.80 mg, about 4.85 mg, about 4.90 mg, about 4.95 mg, about 5.00 mg, about 5.05 mg, about 5.10 mg, about 5.15 mg, about 5.20 mg, about 5.25 mg, about 5.30 mg, about 5.35 mg, about 5.40 mg, about 5.45 mg, about 5.50 mg, about 5.55 mg, about 5.60 mg, about 5.65 mg, about 5.70 mg, about 5.75 mg, about 5.80 mg, about 5.85 mg, about 5.90 mg, about 5.95 mg, about 6.00 mg, about 6.05 mg, about 6.10 mg, about 6.15 mg, about 6.20 mg, about 6.25 mg, about 6.30 mg, about 6.35 mg, about 6.40 mg, about 6.45 mg, about 6.50 mg, about 6.55 mg, about 6.60 mg, about 6.65 mg, about 6.70 mg, about 6.75 mg, about 6.80 mg, about 6.85 mg, about 6.90 mg, about 6.95 mg, about 7.00 mg, about 7.05 mg, about 7.10 mg, about 7.15 mg, about 7.20 mg, about 7.25 mg, about 7.30 mg, about 7.35 mg, about 7.40 mg, about 7.45 mg, about 7.5 mg, about 7.60 mg, about 7.70 mg, about 7.80 mg, about 7.90 mg, about 8.00 mg, about 8.10 mg, about 8.20 mg, about 8.30 mg, about 8.40 mg, about 8.50 mg, about 8.60 mg, about 8.70 mg, about 8.80 mg, about 8.90 mg, about 9.00 mg, about 9.10 mg, about 9.20 mg, about 9.30 mg, about 9.40 mg, about 9.50 mg, about 9.60 mg, about 9.70 mg, about 9.80 mg, about 9.90 mg, about 8.00 mg, about 8.10 mg, about 8.20 mg, about 8.30 mg, about 8.40 mg, about 8.50 mg, about 8.60 mg, about 8.70 mg, about 8.80 mg, about 8.90 mg, about 9.00 mg, about 9.25 mg, about 9.50 mg, about 9.75 mg, about 10.00 mg, about 10.25 mg, about 10.50 mg, about 10.75 mg, about 11.00 mg, about 11.25 mg, about 11.50 mg, about 11.75 mg, about 12.00 mg, about 12.25 mg, about 12.50 mg, about 12.75 mg, about 13.00 mg, about 13.25 mg, about 13.50 mg, about 13.75 mg, about 14.00 mg, about 14.25 mg, about 14.50 mg, about 14.75 mg, and about 15.00 mg.

Derivatives prepared using aspirin, citric acid, sodium bicarbonate, L-theanine cocrystal composition according to embodiments of the invention can be administered via the sublingual route.

The pharmaceutical compositions according to embodiments of the invention may be prepared as oral disintegrating tablets, oral liquids, quick dissolves, granules, wafers (films), pellets, or powders.

Embodiments of the invention includes water soluble excipients with optimal disintegration properties.

Cocrystals according to embodiments of the invention may be used to improved one or more physical properties, such as solubility, stability, and dissolution rate, of the active pharmaceutical ingredient of a selected treatment or prevention.

The invention is described in further detail by means of examples, without intending to limit the scope of the invention to these examples alone. While a specific embodiment of the invention has been shown and described in detail to illustrate the application of the principles of the invention, it will be understood that the invention may be embodied otherwise without departing from such principles.

Theanine cocrystals, including aspirin/theanine cocrystals, are described in U.S. Pat. Nos. 9,603,937, 9,603,938, 9,603,939, 9,289,438, 9,289,439, 9,289,440, 8,685,948, 8,476,250, 8,304,404, and 8,173,625, which are incorporated herein by reference in their entirety.

### REFERENCES

1. “Heart Disease and Stroke Statistics 2017 At-a-Glance.” https://www.heart.org/idc/groups/ahamah-public/@wcm/@sop/@smd/ . . . N.p., n.d. Web.

2. Virmani, Renu. “Pathophysiology of Acute Myocardial Infarction.” Medical Clinics of North America, by Allen P. Burke, Blackwell, 2007, pp. 88-89.

3. Liu, Jiaqi, et al. “Inflammation and Inflammatory Cells in Myocardial Infarction and Reperfusion Injury: A Double-Edged Sword,” Clinical Medicine Insights: Cardiology, vol. 10, 2016, doi:10.4137/cmc.s33164.

4. PDR for Nutritional Supplements 2nd. ed. Thomson Reuters, Montvale, N.J. 2008, p. 366.

5. “FDA; GRAS Assessment of L-Theanine (98%).” http://www.accessdata.fda.gov/scripts/fcn/gras_notices/GRN000338.pdf. N.p., 5 May 2010.

6. Gomez-Ramirez, Manuel, et al. “The Deployment of Intersensory Selective Attention: A High-density Electrical Mapping Study of the Effects of Theanine”: Clinical Neuropharmacology.” LWW. Clinical Neuropharmacology: January/February 2007—Volume 30—Issue 1—pp. 25-38, n.d. Web. 21 Apr. 2017.

7. “L-Theanine” http://www.chemicalbook.com/ChemicalProduct Property_EN_CB1485976.htm.

8. Jaiswal, Manoj, et al. “Impairment of Mitochondrial Calcium Handling in a mtSOD1 Cell Culture Model of Motoneuron Disease.” BMC Neuroscience, vol. 10, no. 1, 2009, p. 64., doi:10.1186/1471-2202-10-64.

9. Manev, H.; Favaron, M.; Guidotti, A.; and Costa, E., Delayed increase of Ca²⁺ influx elicited by glutamate: role in neuronal death. Molecular Pharmacology. 1989 Jul.; 36(1):106-112).

10. Yokogoshi, Hidehiko; Kobayashi, Miki; Mochizuki, Mikiko; and Terashima, Takehiko, “Effect of Theanine, Glutamylethylamide, on Brain Monoamines and Striatal Dopamine Release in Conscious Rats.” Neurochemical Research, May 1998, Volume 23, Issue 5, pp. 667-673).

11. Alternative Medicine Review, Vol. 10, Number 2, p. 136, 2005, Thorne Research Inc.

12. Sadzuka, Yasuyuki, et al. “Enhancement of the Activity of Doxorubicin by Inhibition of Glutamate Transporter.” Toxicology Letters, vol. 123, no.-2-3, 2001, pp. 159-167., doi:10:10116/s0378-4274(01)00391-5.

13. Peat, Ray. “Aspirin, Brain, and Cancer.” Serotonin: Effects in Disease, Aging and inflammation, raypeat.com/articles/aging/aspirin-brain-cancer.shtml.

14. BTIscience. “Aspirin Targets Key Protein in Neurodegenerative Disease.” EurekAlert!, 30 Nov. 2015, www.eurekalert.org/pub_realeases/2015-11/btif-atk112515.php.

15. Choi, Hyong Woo, et al. “Human GAPDH Is a Target of Aspirinas Primary Metabolite Salicylic Acid and Its Derivatives.” Plos One, vol. 10, no. 11, 2015, doi:10,1371/journal.pone.0143447.

Each reference cited above is hereby incorporated in its entirety as if fully set forth herein.

1. A composition comprising aspirin, citric acid, sodium bicarbonate, and L-theanine.

2-6. (canceled)

7. The composition of claim 1, wherein aspirin, citric acid, sodium bicarbonate, and L-theanine are in the form of a cocrystal.

8. A dosage form comprising the composition of claim 1.

9. The dosage form of claim 8, wherein the dosage form is an oral disintegrating tablet or an amount of powder.

10. A method of treating acute myocardial infarction in a subject in need thereof, comprising administering sublingually to the subject a cocrystal aspirin, citric acid, sodium bicarbonate, and L-theanine sublingual formulation.

11. The method of claim 10, wherein the aspirin, citric acid, sodium bicarbonate, L-theanine cocrystal are absorbed in the subject's bloodstream via the sublingual route and bypass the hepatic first pass effect.

12. The composition of claim 1, wherein the wt.% of aspirin in the composition is between about 20% and about 30%.

13. (canceled)

14. The composition of claim 1, wherein the wt.% of L-theanine in the composition is between about 20% and about 30%.

15. (canceled)

16. The composition of claim 1, wherein the wt.% of sodium bicarbonate in the composition is between about 15% and about 25%.

17. (canceled)

18. The composition of claim 1, wherein the wt.% of citric acid in the composition is between about 5% and about 15%.

19. (canceled)

20. The composition of claim 1, further comprising one or more of a binder, an emulsifier, and a disintegrant.

21. The composition of claim 1, further comprising polyvinylpyrrolidone.

22. (canceled)

23. The composition of claim 21, wherein the polyvinylpyrrolidone is cross-linked.

24. (canceled)

25. The composition of claim 1, further comprising a sugar alcohol.

26-27. (canceled)

28. The composition of claim 1, further comprising a lubricant.

29-30. (canceled)

31. The dosage form of claim 8, wherein the amount of aspirin in the dosage form is between about 300 mg and about 450 mg.

32. (canceled)

33. The dosage form of claim 8, wherein the amount of L-theanine in the dosage form is between about 300 mg and about 450 mg.

34. (canceled)

35. The dosage form of claim 8, wherein the amount of sodium bicarbonate in the dosage form is between about 200 mg and about 350 mg.

36. (canceled)

37. The dosage form of claim 8, wherein the amount of citric acid in the dosage form is between about 75 mg and about 200 mg.

38. (canceled)

39. The dosage form of claim 8, further comprising one or more of a binder, an emulsifier, and a disintegrant.

40-50. (canceled)
